(19) United States
(12) Patent Application Publication
(10) Pub. No.: US 2022/0338837 A1
(43) Pub. Date: Oct. 27, 2022

(54) SCAN NAVIGATION

(71) Applicant: CANON MEDICAL SYSTEMS CORPORATION, Otawara-shi (JP)

(72) Inventors: Robin STEEL, Edinburgh (GB); Chris MCGOUGH, Edinburgh (GB); Gaurav PHADKE, Edinburgh (GB); Tony LYNCH, Edinburgh (GB)

(73) Assignee: CANON MEDICAL SYSTEMS CORPORATION, Otawara-shi (JP)

(21) Appl. No.: 17/241,288

(22) Filed: Apr. 27, 2021

(51) Int. Cl. A61B 8/00; A61B 8/13; A61B 90/00

(52) U.S. Cl. CPC A61B 8/4254; A61B 8/461; A61B 8/13; A61B 90/39; A61B 2090/3925

(57) ABSTRACT

A method of guiding performance of an ultrasound imaging procedure, comprising: detecting at least a position of an ultrasound probe; determining a position of an imaging region of the ultrasound probe relative to a target based on the at least detected position of the ultrasound probe; displaying a figure representative of an imaging region together with a reference image associated with the target wherein the appearance of at least part of the figure is dependent on at least the determined position of the imaging region relative to the target; and updating the appearance of at least part of the figure as the ultrasound probe moves relative to the target.

### FIELD

Embodiments described herein relate generally to a method and apparatus for guiding performance of a medical imaging procedure, for example, a method and apparatus for guiding an ultrasound probe.

### BACKGROUND

Accurate operation of a medical imaging probe, for example, an ultrasound probe, requires a degree of expertise and training, to identify planes to be scanned during a medical imaging procedure without guidance.

Known methods of guiding performance of a medical imaging procedure include guiding based on an image analysis of the ultrasound data from a current exam to decide to determine what part of an anatomy is being imaged.

### BRIEF DESCRIPTION OF THE DRAWINGS

Embodiments are now described, by way of non-limiting example, and are illustrated in the following figures, in which:—

FIG. 1 is a schematic illustration of an apparatus in accordance with an embodiment;

FIGS. 2(a) and 2(b) are schematic illustrations of an imaging region and a target in accordance with an embodiment;

FIGS. 3(a) and 3(b) are schematic illustrations of a display of an apparatus in accordance with an embodiment;

FIG. 4 is a flow chart illustrating in overview a method of guiding an ultrasound probe in accordance with an embodiment;

FIG. 5 is a schematic illustration of further indicators, in accordance with an embodiment;

FIG. 6 is a flow chart illustrating in overview a method of performing medical imaging procedure, in accordance with an embodiment, and

FIG. 7 is a flow chart illustrating in overview a method of performing medical imaging procedure, in accordance with a further embodiment.

### DETAILED DESCRIPTION

Certain embodiments provide a method of guiding performance of an ultrasound imaging procedure, comprising: detecting at least a position of an ultrasound probe; determining a position of an imaging region of the ultrasound probe relative to a target based on the detected position of the ultrasound probe; displaying a figure representative of an imaging region together with a reference image associated with the target wherein the appearance of at least part of the figure is dependent on at least the determined position of the imaging region relative to the target; and updating the appearance of at least part of the figure as the ultrasound probe moves relative to the target.

Certain embodiments provide an apparatus comprising processing circuitry configured to: receive position data representative of a position of an ultrasound probe; determine a position of an imaging region of the ultrasound probe relative to a target based on the received position data; display a figure representative of the imaging region together with a reference image associated with said target, wherein the appearance of at least part of the figure is dependent on at least the determined position of the imaging region relative to the target; and update the appearance of at least part of the figure as the ultrasound probe moves relative to the target.

Certain embodiments relate to a computer program product comprising computer-readable instructions that are executable to: receive position data representation of at least a detected position of an ultrasound probe; determine a position of an imaging region of the ultrasound probe relative to a target based on the detected position of the ultrasound probe; display a figure representative of an imaging region together with a reference image associated with the target wherein the appearance of at least part of the figure is dependent on at least the determined position of the imaging region relative to the target; and update the appearance of at least part of the figure as the ultrasound probe moves relative to the target.

The method and apparatus described herein relate to guiding performance of a medical imaging procedure, for example, a method and apparatus for guiding an ultrasound probe, for example, to image a target (for example, a desired anatomy, feature or imaging plane/region). In the embodiments described in the following, target data are obtained prior to the current scan, including, for example, target data obtained as part of a prior examination process or as part of previously performed analysis is used as part of the guiding process. In other embodiments, the target data are obtained at a prior time during the current exam. By providing the guiding method and apparatus describe in the following, novice or consumer users may be able to obtain ultrasound (US) images of target anatomy as easily and reproducibly as expert users.

Embodiments described herein may have applications in a number of different settings. Non-limiting examples of use cases where probe guidance method and apparatus can be used include: a follow-up scan to reimage exactly the same plane imagined in a prior scan e.g. for assessing lesion growth/shrinkage; imaging ‘standard’ planes such as the standard ultrasound cardiac views e.g. for assessing ventricular volume/function; imaging specific planes requested by a referring clinician—these requested planes may be marked up by them using a workstation on a prior CT/MR/US volume; and imaging regions of interest that are marked-up on the reference volume within the same exam.

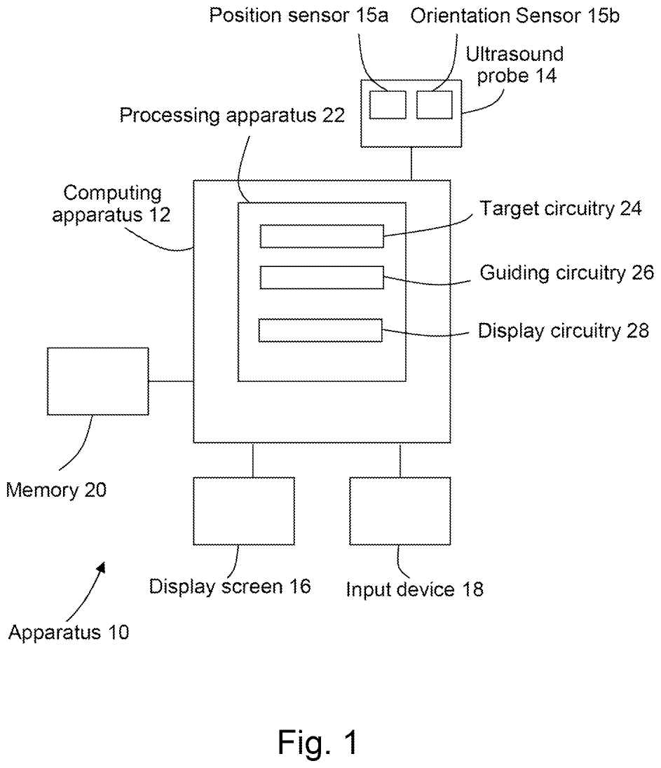

An apparatus 10 according to an embodiment is illustrated schematically in FIG. 1. The apparatus 10 is configured to acquire ultrasound data during an ultrasound scan and to process the ultrasound data to obtain an ultrasound image.

In the present embodiment, the apparatus 10 comprises a computing apparatus 12 and associated ultrasound probe 14. Any suitable type of ultrasound probe 14 may be used. For brevity, the ultrasound probe may simply be referred to as a probe 14. In other embodiments the apparatus 10 may comprise a scanner apparatus of an alternative modality. In the present embodiment, the ultrasound probe 14 has a position sensor 15a and an orientation sensor 15b. As the probe 14 is moved, the position sensor 15a and orientation data sensor 15b provide position data and orientation data representative of the position and orientation of the probe 14. The position data and orientation data are provided to the processing apparatus 22. It will be understood that, while in the present embodiment, the position and orientation are detected by sensors 15a, 15b of the probe 14, in other embodiments, the position and orientation of the probe are detected by sensors provided remotely from the probe.

The apparatus 10 comprises a display screen 16 for displaying a reference image associated with or corresponding to a target to be imaged. The display screen 16 may also be referred to as the display, for brevity. In the present embodiment, the reference image is an image of a target region. In the present embodiment, the display screen 16 also displays a figure together with the reference image to provide guidance to an operator of the probe 14. In the present embodiment, the position and/or orientation of the figure relative to the reference image is representative of the position and/or orientation of the imaging region relative to the target. In the present embodiment, the figure is displayed as an overlay on the reference image such that part of the reference image can be seen through the figure. In some embodiments, the display screen also displays the presently scanned ultrasound image from the probe. In further embodiments, a further display screen is provided for displaying the ultrasound image and/or one or more further indicators separately from the reference image/figure.

The apparatus 10 also comprises an input device 18, provided separately from the probe 14. The input device 18 can be used to provide instructions to the apparatus 10, for example, the input device 18 can be used to indicate to the apparatus 10 that the desired target image has been captured and/or to instruct the apparatus 10 to move onto the next target.

The computing apparatus 12 comprises a processing apparatus 22 for processing of data, including image data. The processing apparatus 22 comprises a Central Processing Unit (CPU) and Graphical Processing Unit (GPU). The processing apparatus 22 includes target circuitry 24, guiding circuitry 26 and display circuitry 28. The circuitries be implemented in the CPU, in the GPU, or in a combination of the CPU and the GPU.

In the present embodiment, the various circuitries are each implemented in the CPU and/or GPU of processing apparatus 22 by means of a computer program having computer-readable instructions that are executable to perform the method of the embodiment. However, in other embodiments each circuitry may be implemented in software, hardware or any suitable combination of hardware and software. In some embodiments, the various circuitries may be implemented as one or more ASICs (application specific integrated circuits) or FPGAs (field programmable gate arrays).

In alternative embodiments the processing apparatus 22 may be part of any suitable scanning apparatus (for example a CT scanner or MR scanner) or image processing apparatus (for example, a PC or workstation). The processing apparatus 22 may be configured to process any appropriate modality of imaging data.

In some embodiments, different circuitries are implemented in different apparatuses. For example, in some embodiments, the display circuitry 28 is implemented in a further computing apparatus, for example a PC or workstation that does not form part of the computing apparatus 12.

The processing apparatus 22 also includes a hard drive and other components including RAM, ROM, a data bus, an operating system including various device drivers, and hardware devices including a graphics card. Such components are not shown in FIG. 1 for clarity.

The system of FIG. 1 is configured to perform a method of guiding performance of an ultrasound imaging procedure, in accordance with an embodiment. The method includes providing guidance to an operator of the probe 14 during an imaging procedure via the display screen 16.

In further embodiments, the display screen is provided as part of the probe itself. In other embodiments, more than one display screen is provided, for example, a display screen on the probe is provided together with the display screen. In such embodiments, the figure and reference image are displayed on a first display screen and one or more further indicators (for example, inset views or image similarity indicators) and/or a present ultrasound image are provided on the second display screen.

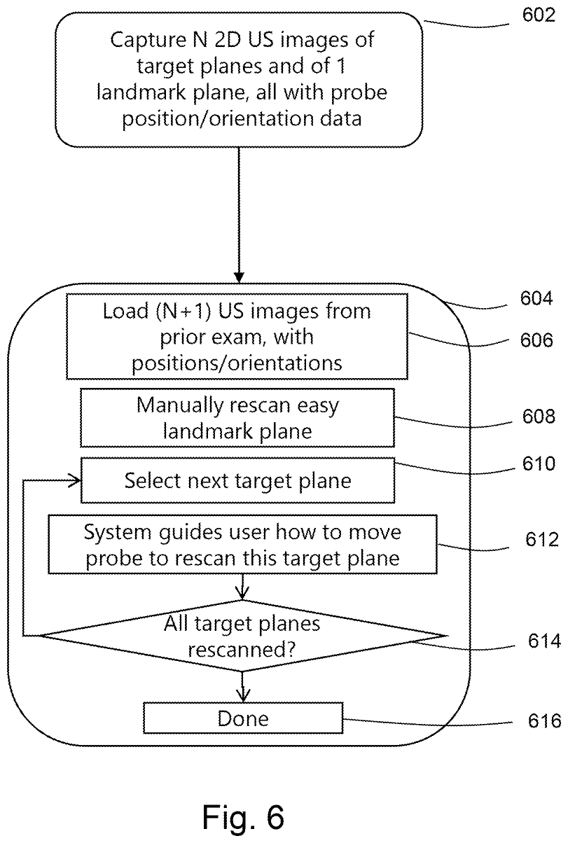

In the present embodiment, the circuitries of the processing apparatus 22 operate as follows. The target circuitry 24 obtains target data, for example, target data that has been previously collected. Further detail on the acquisition of the target data is provided with reference to FIG. 6. The target data may be retrieved from storage on memory 20 or may be accessed from a further device. The target data comprises target image data representative of one or more desired targets to be imaged (as part of an imaging procedure or imaging protocol) and position and orientation data associated with the targets. For each target, the target image data is processed by display circuitry 28 to generate a reference image corresponding to the target region. The reference image is then displayed on the display screen 16. For each target, the guiding circuitry 26 processes the target position and target orientation data for the target together with the current position data and orientation data received from the position sensor 15a and the orientation sensor 15b to determine values for one or more guide parameters. The guiding circuitry 26 may also process the image data itself to determine guide parameters. The one or more guide parameters relate to, for example, the shape/position/colour/texture of one of more visual aspects of the guide. The display circuitry 28 receives these values and displays the visual guide, in accordance with these parameters, on the display screen 16 together with the reference view.

The position/orientation data for the probe 14 and for the targets are measured relative to a reference. In the present embodiment, this reference corresponds to a landmark image. However, it will be understood that other reference frames and/or reference points can be used.

In the present embodiment, the guide displayed on the display screen 16 includes a figure that overlays the reference view. This is displayed together with a further indicator in the form of a fine inset view and/or a coarse inset view. The values of the guide parameters and therefore the appearance of the guide (the figure and the further indicators) are dependent on at least a measure of distance between the imaging region being imaged by the probe 14 and the target.

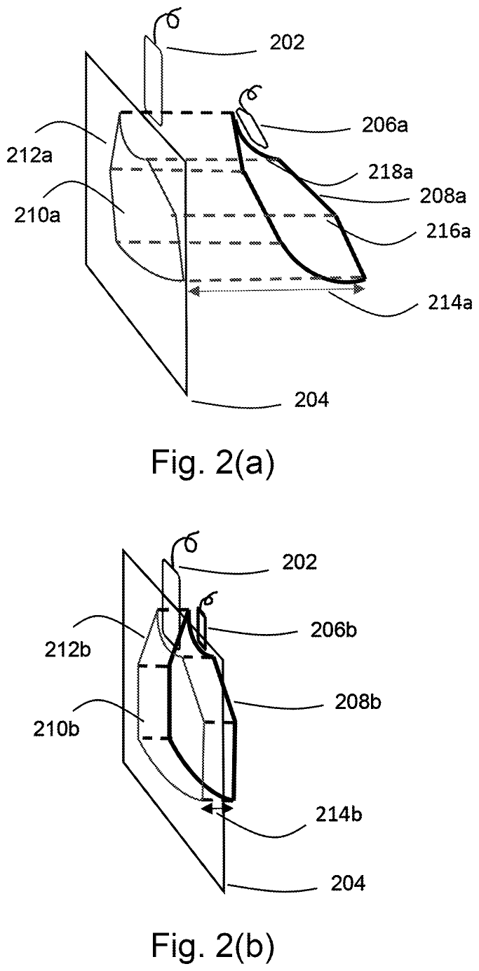

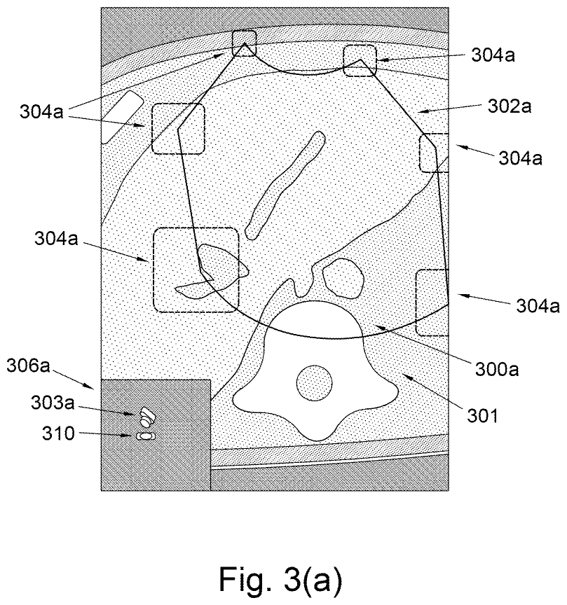

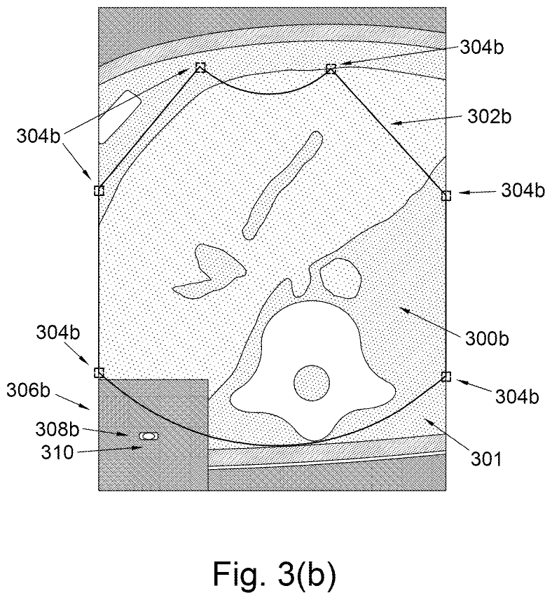

FIGS. 2(a), 2(b), 3(a) and 3(b) illustrate how the appearance of the guide, in particular the figure overlaying the reference view, relates to the position of the imaging region and/or probe 14 relative to the target. FIG. 2(a) illustrate a first spatial relationship between a first imaging region 208a and a target and FIG. 3(a) depicts the corresponding displayed view, in accordance with the present embodiment. FIGS. 2(b) and 3(b) illustrate a second spatial relationship between a second imaging region 208b and the target and the corresponding displayed view, respectively, in accordance with the present embodiment. The second spatial relationship corresponds to the imaging region being closer to the target than the first spatial relationship.

Turning to FIG. 2(a) and FIG. 2(b) a target corresponding to a target probe position 202 and a target plane 204 is depicted (for brevity, the target probe position 202 may be referred to simply as the target position). It will be understood that a target plane can be determined using a target probe position and a target probe orientation. Data representative of the target position/orientation forms part of the target data. FIG. 2(a) also depicts a first probe position 206a and a corresponding first imaging region 208a. Likewise, FIG. 2(b) depicts a second probe position 206b and corresponding second imaging region 208b. It will be understood that the first/second imaging regions can be considered to lie in first/second imaging planes and are therefore dependent on both the probe position and orientation.

The first and second probe positions 206a, 206b and corresponding first and second imaging regions 208a, 208b are examples of probe positions at a first and second time. The first and second probe position 206a, 206b and the first and second imaging regions 208a, 208b can therefore be understood as instances of a current or live probe position and a current or live imaging region. It will be further understood that movement of the probe from a current probe position/orientation will cause a change in the corresponding current imaging region. An imaging region for the probe 14 (for example, the first and second imaging regions) is determined by processing position and orientation data representative of the position and orientation of the probe 14. Differences in position and/or orientation can be calculated using different methods. For example, a distance between the target and the imaging region can be calculated by subtracting one position from another. As a further example, a difference in orientation can be calculated by determining an angle between the target plane and imaging plane or other related point/line of reference. Orientation may be represented by more than one angle. In one non-limiting example, an angle between a normal of the target plane and a normal of the imaging plane. In addition, a further angle may represent rotation about the normal.

The first imaging region 208a can be considered to be in a first spatial relationship with the target, for example with the target plane 204. Likewise, the second imaging region 208b can be considered to be in a second spatial relationship with the target, for example with the target plane 204. Such a spatial relationship can be characterized by one or more distances measured between different point of the imaging region and the target plane 204.

For example, FIG. 2(a) illustrates three such distances: a first distance 214a, a second distance 216a and a third distance 218a. A corresponding first distance 214b is depicted in FIG. 2(b). It will be understood that other distances can be calculated between points of the imaging region and the target plane 204 (that are not shown in FIGS. 2(a) and 2(b) for clarity). Determining distances from different points on the imaging region allows the guide to convey additional distance information local to these points.

With reference to FIG. 2(a), in use, the first imaging region 208a is projected onto the target plane 204 to form a first projected region 210a. The first projected region 210a lies in the target plane 204 and is defined by a first boundary 212a. As depicted in FIG. 3(a), a first FIG. 300a that has an outline corresponding to the first boundary 212a is overlaid on the reference image 301 during guidance of the ultrasound probe 14. The appearance of the figure (for example, the shape/colour/form) provides guidance information for the operator of the probe 14 as they navigate the probe 14 to scan the target. The first FIG. 300a is representative of the position of the first imaging region 208a relative to the target. While first boundary 212a is represented in FIG. 2(a) as a black line, it will be understood that, in the present embodiment, this line is green. The appearance of the at least part of the figure may convey, visually, guidance information.

With reference to FIG. 2(b), in use, the second imaging region 208b is projected onto the target plane 204 to form a second projected region 210b. The second projected region 210b lies in the target plane 204 and is defined by a second boundary 212b. As depicted in FIG. 3(b), a second FIG. 300b that has an outline corresponding to the second boundary 212b is overlaid on the reference image 301 during guidance of the ultrasound probe 14. The second FIG. 300b therefore provides guidance information for the operator. In particular, the second FIG. 300b is representative of the position of the second imaging region relative to the target. While second boundary 212b is represented in FIG. 2(b) as a black line, it will be understood that, in the present embodiment, this line is red.

As described above, FIGS. 3(a) and 3(b) illustrate generated displays for guiding operation of the ultrasound probe 14. In the present embodiment, the appearance of the generated displays are in accordance with values of one of more guide parameters that are selected dependent on the determined position/orientation of the imaging region relative to the target. The guiding parameters and thus appearance of the guide, in particular, the figure and further indicators are updated in response to movement of the probe.

FIG. 3(a) is a screenshot of a first display corresponding to FIG. 2(a). FIG. 3(a) depicts the first FIG. 300a, described above, which in the present embodiment, is a fan representation. The first FIG. 300a is represented by a first boundary line 302a that corresponds to the first boundary 212a of the first projected region 210a. While first boundary line 302a is represented in FIG. 3(a) as a black line, it will be understood that, in the present embodiment, first boundary line 302a is a green line. The first FIG. 300a also has a number of corner markers 304a positioned at corners of the first boundary line 302a. While corner markers 304a are represented as black dashed lines in FIG. 3(a), it will be understood that, in the present embodiment, the corner markers 304a are green. In the present embodiment, the corner markers 304a are provided at discontinuities in the boundary line. The first FIG. 300a is overlaid on a reference image 301 that corresponds to the target to be imaged.

Likewise, FIG. 3(b) depicts a screenshot of a second display corresponding to FIG. 2(b). FIG. 3(b) depicts the second FIG. 300b. The second FIG. 300b is also a fan representation. The second FIG. 300b is represented by a second boundary line 302b that corresponds to the second boundary 212b of the second projected region 210b. While second boundary line 302b is represented in FIG. 3(b) as a black line, it will be understood that, in the present embodiment, second boundary line 302a is a red line. The second FIG. 300b also has a number of corner markers 304b positioned at corners of the second boundary line 302b. While corner markers 304b are represented as black dashed lines in FIG. 3(b), it will be understood that, in the present embodiment, the corner markers 304b are red. The second FIG. 300b is overlaid on a reference image 301 that corresponds to the target to be imaged.

While a first FIG. 300a and a second FIG. 300b are described, it will be understood that these can be considered as different instances of the same figure. In use, as the probe 14 is moved from the first probe position 206a of FIG. 2(a) to the second probe position 206b of FIG. 2(b), the appearance of a single figure is continuously updated, in real time, in response to the movement of the probe 14 while the reference image 301 remains static or fixed. Therefore, the displayed figure will, take on the appearance of the first FIG. 300a when the probe 14 is at the first probe position 206a and take on the appearance of second FIG. 300b when the probe is at the second probe position 206b.

FIGS. 3(a) and 3(b) depict displayed figures with visual aspects that are defined by a first set of characteristics (i.e. the shape and/or colour of the boundary line and/or the size of markers) for two different imaging regions. It will be understood that, as the imaging region is changed (via movement of the probe 14) the visual aspects will be updated. In particular, characteristics of the visual markers will change continuously, in real-time in response to a change in the imaging region. The shape of the projected regions will change as the probe position/orientation changes relative to the target and therefore the displayed shape of the figures will therefore change, accordingly. The change in shape is a continuous change of shape as the probe 14 is moved relative to the target.

The colour of the figure (including boundary line and corner markers) also provide a continuously varying visual aspect of the figure that changes as the probe 14 is moved. For example, in some embodiments, the colour of the boundary line is a particular colour, for example red, or shade of a particular colour or has a particular brightness. The colour and/or shade of the colour and/or brightness of the colour varies as the probe 14 moves and conveys information about the position/orientation of the imaging region relative to the target. The colour and/or shade and/or brightness is therefore representative of the distance between the imaging region and the target.

In the present embodiment, the colour of the figure is provided on a colour scale between two colours, in particular between red and green. In the present embodiment, movement of the probe 14 changes the colour along the scale from red to green. In particular, the colour varies between red when the probe is further from the target to green when the probe is closer to the target. The colour scale can be defined by a hex code or other numerical representation of a colour, for example, a RGB colour code. The colour change may be considered as corresponding to the colour becoming redder as the probe moves further from the target e.g. if the colour is represented by a RGB colour code the R component of the RGB colour code increases in size relative to the G and B components. The colour change may be considered as corresponding to the colour becoming greener as the probe moves closer to the target e.g. the G component of the RGB colour code increases in size relative to the R and B components. While red and green are used in the present embodiment, it will be understood that other colours can be used in other embodiments. In the present embodiment, the colour varies between green and red locally in dependence on a local distance between the target and the imaging region. The local distance may be a perpendicular distance between the imaging region and the target.

In other embodiments, as the probe 14 moves closer to the target position, the shade of the selected colour, for example, red, becomes brighter or more vivid and as the probe 14 moves further from the target position, the shade of the selected colour becomes duller or less vivid. As a further non-limiting example, the colour of the figure is provided on a colour scale between two shades of a colour such that movement of the probe 14 changes the colour along the scale (for example, the colour may vary between a dark red, when the probe is further from the target and a bright red, when the probe is close to the target). The colour scale can be defined by a hex code or other numerical representation of a colour.

In the present embodiments, the continuous change of colour is between two colours, namely between red and green, such that when the imaging region is far from the target the colour is red and as the imaging region approaches the target, the imaging region turns green. In the present embodiment, in FIG. 3(a) the colour of the first FIG. 300a is red and in FIG. 3(b) the colour of the second FIG. 300b is green, to indicate that the probe 14 is aligned with the target.

In the above-described embodiments, a continuous change of colour is described. In other embodiments, a discrete step change in colour occurs when the imaging region is substantially close to the target. In some embodiments, the boundary line turns to a different colour in response to the imaging region being close to the target plane, for example, if the probe 14 position and/or imaging plane is substantially close to the target position and/or target plane. In such embodiments, the boundary line may be a shade of red (that varies continuously and/or locally in dependence on distances between the imaging region and the target) that turns green in response to being substantially close.

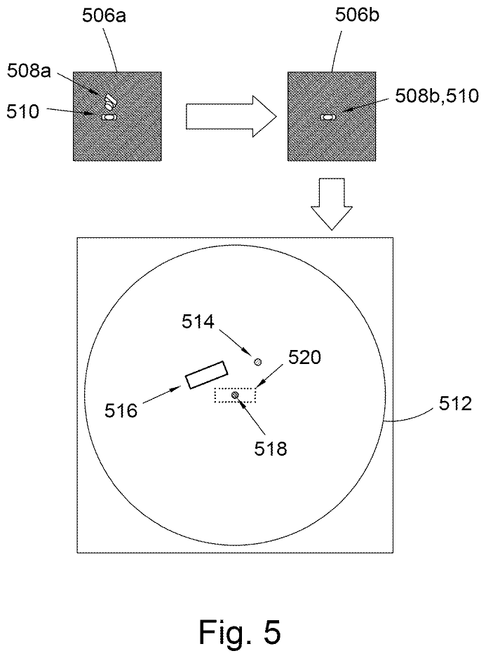

A number of actions may be performed in response to determining that the imaging region is substantially close to the target. For example, a screenshot may be taken automatically, or the inset view may be modified (as described with reference to FIG. 5). It will be understood that in this context, substantially close may correspond be a distance being below a pre-determined threshold which may be dependent on parameters/tolerance of the system being used and/or the application being used. For example, if the positioning system has a tolerance of the order of 1.4 mm RMS or 0.5 degrees RMS, a threshold above this value may be used. In some embodiments, the maximum distance between the imaging plane and the target may be also be displayed.

As a second example of a continuously varying visual aspect of the figure, the size of the corner markers are dependent on the distance(s) between the imaging region and target. This visual aspect allows orientation information to be depicted in a graphical form as each corner marker can be a different size depending on its particular distance to the target plane, in this case a perpendicular distance to the target plane. For example, if the probe position is maintained but the orientation of the probe is changed, then the one or more corner marker would change size. The operator, aiming to reach a target would therefore, in the present embodiment, aim to move the probe 14 to change the colour to a uniform green and also aim to decrease the corner markers in size. When aiming to change the colour to uniform green the figure including boundary lines and corner markers will start red and change towards a greener colour as the imaging region is moved closer to the target plane. If a particular corner marker is larger than the others, then an operator is aware that an appropriate change in orientation is required.

While size of the corner markers are described as changing size based on the local perpendicular distance to the target plane, other visual aspects may also vary continuously based on the local perpendicular distance to the target plane, for example, the colour may change continuously along the perimeter. Such visual aspects may be changed locally in combination. For example, corner boxes and the colour of local parts of the boundary may both change in dependence on the perpendicular distance between the local part of the boundary and the target plane.

In general, when the imaging region is at a further distance from the target, for example, as illustrated in FIG. 3(a), the corner markers 304a and boundary of the figure are redder in colour relative to when the imaging region is at a closer distance from the target. In addition, the corner markers 304a are larger than when the imaging region is at a closer distance from the target. The colour is redder relative to a greener colour figure corresponding to a closer distance. Furthermore, in the inset view, the probe representation and the target representation will be further apart than when the imaging region is closer to the target.

Square markers are described in the above embodiments, however, it will be understood that, in other embodiments, the markers may be rectangular and/or circular or take the form of the error bar.

In general, at a closer distance to the target, for example, as illustrated in FIG. 3(b), the corner markers 304b are smaller. In addition, when the imaging region is at a closer distance from the target the corner markers 304a and figure turn a greener colour relative to when the imaging region is at a further distance. When the distance between the imaging region and target is below a threshold value the figure including markers and boundary has turned green and the size of the markers substantially vanish.

As can be seen in FIG. 3(a), in the present embodiment, each corner marker has a size dependent on a distance between the imaging region and the target plane 204. In particular, the distance information is used to determine a size/shape/colour of the figure. As described below, corner markers 304a are depicted in FIG. 3(a). These corner markers 304a are drawn at corners of the figure corresponding to discontinuities in the boundary and each has a size dependent on the corresponding distance measured from the corner of the imaging region to the target plane 204. The variation in these distances will be dependent on, for example, the relative orientation of the current imaging plane and the target plane 204 and/or the current position of the probe 14 relative to the target probe position 202. For example, when the imaging plane and the target plane 204 are parallel then the distances between different parts of the imaging region and their corresponding part on the target plane 204 will be substantially equal. In some embodiments, the largest distance between the imaging plane and the target is displayed.

In more detail, with reference to FIG. 2(a), as the first distance 214a is larger than the second distance 216a, the corner marker corresponding to the corner from where the first distance 214a is measured will be larger than the corner marker corresponding to the corner from where the second distance 216a is measured.

Therefore, each corner marker conveys distance/orientation information for that part of the imaging region. As some corner markers change differently relative to other corner markers, it will be understood that only part of the figure changes appearance as the probe 14 is moved. For example, the boundary line may have different colours corresponding to different distances. In other embodiments, other parts of the figure, for example, the boundary, can change as the probe 14 is moved.

In addition to guidance provided by the figure and reference image, the guiding method also allows additional guidance to be provided in the form one or more further indicators. These further indicators include inset views and image similarity metrics.

FIG. 3(a) depicts a first inset view 306a displayed together with the FIG. 300a and reference image 301. The inset view is overlaid over the reference image 301 in a separate window. The first inset view 306a shows a probe representation 308a and a target representation 310. As illustrated in FIG. 3(a), a relative position between the current probe and the target is displayed in the first inset view 306a by a distance between the probe representation 308a and the target representation 310. In addition, the probe representation 308a is rendered to appear 3D, the rendering therefore providing the user with additional orientation information. As the probe 14 is moved, the target representation 310 is fixed and the probe representation 308a moves. The target representation 310 has the same appearance as the probe representation 308a when viewed from above (as if the probe representation 308a was at the target).

FIG. 3(b) also displays a second inset view 306b substantially the same as the first inset view 306a described above. However, in contrast to the first inset view 306a, the probe representation depicted in the first inset view (second probe representation 308b) is coincident with the target representation 310 corresponding to the probe being at the target position 202.

In the above described embodiment, the figure represents the boundary or outline of a two-dimensional imaging plane (scan plane) imaged by the probe which is operable to scan in two dimensions. In other embodiments, the figure represents a boundary or outline of the 3D scan volume or scan region scanned by a probe operable to scan in three dimensions.

In the above-described embodiments, a figure having two colours was described (a red colour and a green colour). However, it will be understood that further colours can be used to indicate that the probe and/or imaging region is in front of the target or behind the target. In particular, in such an embodiment, the figure moves between three different colours or shades of colour, such that the first colour or shade (e.g. blue) corresponds to an imaging region infinitely far in front of the target the second colour or shade (e.g. red) correspond to an imaging region infinitely far behind the target and the third colour or shade (e.g. green) corresponds to an imaging region closely aligned with the target. In some three colour embodiments, the figure colour can vary continuously around its perimeter between these three colours based on the distance perpendicular to the display screen, between the live scan plane and the target scan plane. Alternatively, or in addition, the figure can become dashed or dotted when the live plane is flipped relative to the target plane (e.g. such that their normal of the planes are at a mutually obtuse angle).

Likewise, in some embodiments, the corner markers can change shape or form depending on whether the imaging region is in front of or behind the target. For example, in one such embodiment, the marker can switch between solid lined and dashed lined when the imaging region is in front/behind the target.

In some embodiments, a further inset view is displayed in response to the distance between the imaging region and the target being below a pre-determined threshold to assist a user in fine-tuning the position and orientation of the probe relative to the target. Further details regarding the fine-tuning inset view are provided with reference to FIG. 5.

In further embodiments, a measure of image similarity is also displayed. The measure of image similarity is determined by performing an image comparison process between the target image data and the current imaging data and provides additional guidance for an operator. The measure of image similarity can be displayed in real-time and changed in response to movement of the probe 14. The measure of similarity can be based on a simple image metric, or in embodiments, where the image is a ‘standard’ view (for example, a cardiac plane) using a model that has been trained on prior images of the target and surrounding planes. The measure of similarity can be calculated, for example, using a neural network. The image similarity measure may be useful when an operator is close to the target. The measure of image similarity may be represented as a number, a percentage or a point on a scale.

In the above-described embodiments, the further indicators are described as inset views and/or a measure of similarity. It will be understood that such further indicators can be displayed separately from the figure and reference image, for example, on a separate display screen. In some embodiments, the further indicators (the fine/coarse inset views and/or the similarity indicator) are displayed on a display screen of the probe 14 itself.

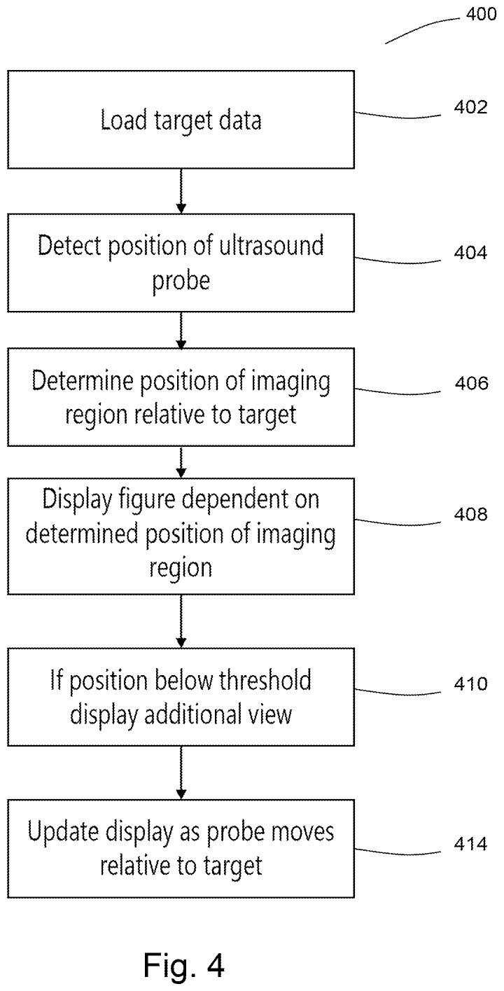

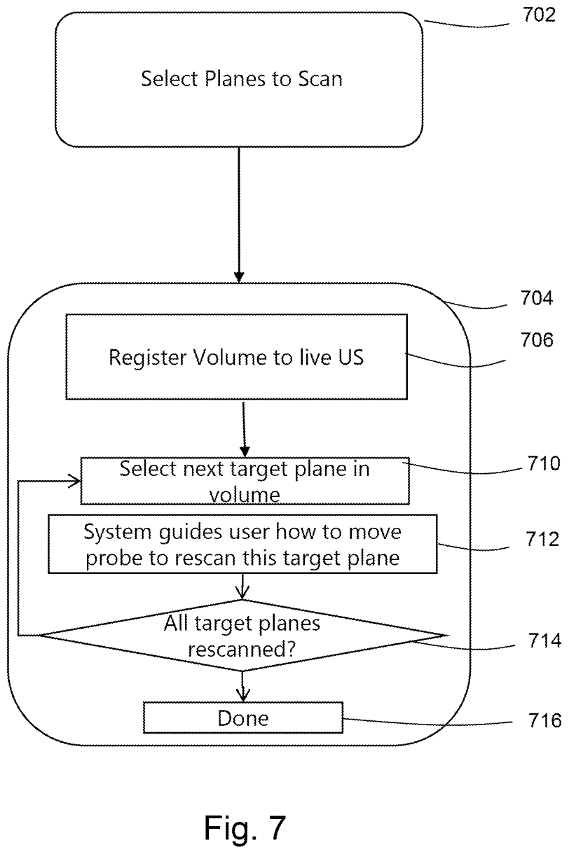

FIG. 4 shows, in overview, a method 400 of guiding the probe 14 using the apparatus described with reference to FIG. 1. At step 402, pre-determined target data associated with a target to be imaged is loaded, by target circuitry 24. Further details on how target data is obtained is provided with reference to FIG. 7. The target data comprises both target image data and target position data and target orientation data.

At step 404, a position of the ultrasound probe 14 is detected. In the present embodiment, the position of the ultrasound probe 14 and the orientation of the ultrasound probe 14 are determined using the probe position sensor 15a and the probe orientation sensor 15b. The position and orientation of the ultrasound probe 14 are monitored throughout the guiding process. The position and orientation are therefore detected continuously as the operator moves the ultrasound probe 14 during the guiding process.

At step 406, a position of the imaging region relative to the target is determined. The position of the imaging region relative to target is determined by processing the received probe position and probe orientation data together with the target position data and target orientation data. It will be understood that the position of the imaging region relative to the loaded target can be determined using a number of different position determining methods. In the present embodiment, a distance between the current probe position (for example, first probe position 206a or second probe position 206b) and the target probe position 202 is determined. In the present embodiment, a current imaging plane is also determined and a mapping between the current scan plane and the target scan plane is determined. The mapping allows a projection of the imaging region (for example, the first projected imaging region 210a and the second projected imaging region 210b) onto the target plane 204 to be determined thereby to allow the projected imaging region to be defined (for example, the first projected imaging region 210a or the second projected imaging region 210b).

Other processing steps can be used to determine the distance. For example, after determining the boundary of the imaging region a number of distances between the imaging region and the target plane 204 may be determined. For example, these distances may include the distance measured from the corners or discontinuities of the imaging region. Once these distances are determined, the projected imaging region or the boundary of the projected imaging region can be determined and then displayed.

At step 408, the figure that is dependent on the determined position of the imaging region is displayed. In the present embodiment, the figure is displayed together with the reference image corresponding to the target. Further detail on the display of the figure and reference image is provided with reference to FIGS. 2(a), 2(b), 3(a) and 3(b). The inset view is also displayed, as described with reference to FIGS. 3(a) and 3(b).

At step 410, a comparison is made between the determined position of the imaging region and the target. If the distance between the imaging position and the target is below a pre-determined threshold value, a fine-tuning inset view is displayed. Further detail relating to the fine-tuning inset is provided with reference to FIG. 5. It will be understood that, in the present embodiment, the coarse inset view is displayed in place of the fine inset view displayed at step 408.

At step 414, the appearance of the display screen 16 is updated as the probe 14 is moved relative to the target. In particular, the figure and inset view(s) are updated as the probe 14 is moved relative to the target. By updating the appearance in real-time, responsive to movement of the probe 14, feedback is provided to the operator to aid in guiding the probe 14 to image the target.

As described with reference to FIGS. 3(a) and 3(b), an inset view is displayed to provide additional guidance to an operator, herein referred to as a coarse inset view. However, it has been found that fine angular adjustment of the probe 14 may be difficult using the coarse inset view. By separating the translation and angular adjustments required on a new inset, a final angular and translational lock on the target may be facilitated. As described in the following, a fine-tuning inset view is therefore also displayed when the distance between the imaging region and the target is below a threshold to provide additional guidance to fine tune position and orientation of the probe 14 to image the target. In the present embodiment, this distance is a translational distance between the probe 14 and target. Should the translational distance between the probe 14 and target become larger than the threshold, the fine inset view switches back to the coarse inset view.

FIG. 5 illustrates a fine-tuning inset view 512 together with two iterations of the coarse inset-view. The first coarse inset view 506a and the second coarse inset view 506b correspond to first inset view 306a and second inset view 306b described with reference to FIGS. 3(a) and 3(b). First coarse inset view 506a and second coarse inset view 506b have corresponding visual elements (probe and target representations) 508a, 508b and 510, corresponding to 308a, 308b and 310.

The fine-tuning inset view 512, also referred to for brevity as the fine inset view 512, has a first probe representation 514 and a second probe representation 516. The fine inset view 512 also has a first target representation 518 and a second target representation 520. In the present embodiment, the first probe representation 514 has the same form as the first target representation 518 (in this case, a dot) and the second probe representation 516 has the same form as the second target representation (in this case, a rectangle). The representations may be displayed in colours, for example, different colours. For example, the first and second target representations may be green.

In the fine inset view the probe orientation representation 516 (the 2D rectangular footprints) is sensitive to angular offset only and show probe angular offset from the North Pole on a polar grid, looking down on the probe 14 from above. In contrast, the probe position representation 514 is sensitive to residual translational offset only.

In contrast to the coarse inset view, the fine inset view does not depict a 3D or rendered representation of the probe 14. Rather, the first probe representation 514 is representative of a probe position and the second probe representation 516 is representative of a probe orientation. Likewise, the first target representation 518 is representative of a target position and the second target representation 520 is representative of a target orientation. The first probe and first target representations can therefore be referred as a probe position representation and a target position representation. Likewise, the second probe and second target representations can therefore be referred as a probe orientation representation and a target orientation representation.

While in FIG. 5, a dot and a rectangle are used for the position and orientation representations, it will be understood that other visual elements and other shapes may be used. In particular, for the orientation representations any shape that can represent an angle of orientation is suitable.

In the present embodiment, the distance in the coarse inset view between the probe position and the target position representations is proportional to a translational distance between the probe 14 and the target probe position 202. However, in other embodiments, other measures of distance between the current probe position and/or imaging region can be represented. In addition, the representation may not be of the probe itself, but, for example, a part of the imaging region (i.e. the closest or furthest part of the imaging region from the target).

It will be understood that, in the present embodiment, the angular distance in the fine inset view between the probe orientation and the target orientation representations is proportional to an angular difference between the orientation of the probe and the target probe orientation. However, in other embodiments, other measures of orientation difference can be represented. In addition, the representations may not be of the probe itself, but, for example, a part of the imaging region (i.e. the closest or furthest part of the imaging region from the target).

In use, when the imaging region is closer to the target than the threshold the fine inset view 512 is displayed. An operator can then refer to the fine inset view to fine tune the probe position and orientation to reach the desired target allowing the desired scan to be performed. In particular, to move the probe 14 into the target position, movement of the probe 14 should be undertaken to reduce the distance, in the fine inset view, between the probe position representation 514 and the target position representation 518 (by translational movement of the probe). In addition, to align the present imaging plane with the target plane 204, movement of the probe 14 should be undertaken to align the probe orientation representation 516 with the target orientation representation 518 (e.g. by rotation of the probe 14).

In other embodiments, rather than switching between the inset views, the relative sizes of the inset view vary dependent on the distance between the imaging region and the target. For example, the coarse inset view may become larger and more prominent as the distance between the imaging region and the target becomes bigger and/or the coarse inset view may become smaller and less prominent as the distance between the imaging region and the target becomes smaller.

As described above, a method of providing guidance to an operator is described which uses target data that is obtained prior to the guiding procedure. FIG. 6 is a flow-chart describing, in overview, a method of performing an ultrasound scanning procedure, using the apparatus of FIG. 1, including the step of acquiring target data representative of a number of target planes to be scanned. FIG. 6 describes a method of a follow-up scan procedure.

At step 602, target data representative of a plurality of target planes is acquired. In the present embodiment, the target data is obtained as part of a previous examination of the subject. It will be understood that, in different embodiments, target data can be acquired using different methods.

In the present embodiment, at step 602 of the method, a prior image acquisition process is performed as part of a prior examination. In the present embodiment, target data is acquired by capturing a set of ultrasound images. The set of reference ultrasound images include a number (N) of two-dimensional ultrasound images of targets planes and one image of a landmark plane. As part of the target data acquisition process, position and orientation data are obtained for each ultrasound image such that each reference ultrasound image data set has a corresponding position and orientation data set. In the present embodiment, the target data is acquired manually, through operating the scanner to scan a set of target planes. However, in other embodiments, target data is acquired using different methods. Further detail regarding different methods of acquiring target data is provided with reference to FIG. 7.

In the present embodiment, the target data is acquired from a previous scanning procedure (for example, a known ultrasound examination procedure. It will be understood that, while ultrasound images are described, a suitable scanning system for obtaining target data during the previous scanning procedure includes any type of medical imaging system that also has the capability to record probe position/orientation during the imaging procedure. As non-limiting example, the prior volumes may be at least one of CT, MR, US, PET volumes.

It will therefore be understood that in some embodiments, the target data including the target image data and target position/orientation image data can be acquired using different methods, for example, using imaging methods of a different modality to that of the imaging procedure being guided. For example, the reference image provided by the target data can be obtained using a different type of scanning procedure (for example, CT, MR, PET).

At step 604 of the method, an ultrasound imaging procedure is performed, at a later time from the time at which step 602 was performed. The ultrasound imaging procedure guides an operator to perform scans of the N target planes acquired during the target data acquisition process.

At step 606, the target data is retrieved. In the present embodiment, the N ultrasound images (the target image data) previously obtained, are loaded together with their position/orientation data (the target probe position and orientation data).

At step 608, an initial scanning step is performed, in which the operator scans the landmark plane. The landmark plane is used as a reference as this plane is relatively easier to scan. This step provides a reference for the subsequent scans. In particular, the landmark scan of the target data provides a reference point for the target position/orientation data of the other targets. Therefore, once the landmark scan is performed, a reference point for subsequent images is provided. For the landmark scan, to assist a user in scanning the landmark plane, a 2D landmark image from the prior scan is displayed together with the live image and the image similarity measure.

At step 610, the next target plane to be scanned is selected and at step 612, guidance is provided to the user on how to scan the target plane. Steps 610 and 612 corresponds substantially to the method of guiding an ultrasound scan (guiding a user to scan a target plane) described with reference to FIG. 4.

In some embodiments, a successful scan may be decided by the operator, and user input therefore provided to the system that is representative that the scan is successful. The determination that a scan is successful may be assisted by the image similarity measure and/or positional/orientation distance between live and target planes. In further embodiments, the success of the scan is determined automatically or by prompting the operator for confirmation, using, for example, the image similarity measure being above a pre-determined threshold and/or the positional/orientation distance between the target and the imaging region being below a pre-determined threshold.

Following a successful scan the method includes a decision step 614 which asks if all target planes have been scanned. If all target planes have not yet been scanned, the method returns to step 610 (select next target plane). If all target planes have been scanned, the method completes at step 616.

Further embodiments in which target data are acquired using different methods are described with reference to FIG. 7. A number of steps of the method of FIG. 7 are substantially the same as the steps described with reference to FIG. 6. For example, the guiding steps: steps 710, 712, 714 and 716 substantially correspond to steps 610, 612, 614 and 616 and are not discussed in further detail.

In the method of FIG. 6, a follow-up scan procedure was described in which target data comprising N target planes and a landmark image was acquired. In that embodiment, the operator manually rescans the landmark image and provides user input to instruct the system that the live image is the same as the landmark. In contrast, the method of FIG. 7 requires targets specified on a reference 3D volume (represented by 3D volume data) rather than N 2D planes. In this embodiment, at step 702, target data is acquired by selecting N desired planes of the reference 3D volume to scan. The following non-limiting examples of selecting desired planes are described.

As a first non-limiting example, the target data is acquired from a previous scanning procedure (for example, a known ultrasound examination procedure). During that scanning procedure, the sonographer marks N target regions of interest that they would like to scan at a later date. As a second non-limiting example, the target data is acquired by marking up, a previously acquired scan volume (for example, by CT, MR, US or PET) with target planes to be scanned during an ultrasound procedure. As a third non-limiting example, target data is generated by an algorithm that automatically determines a set of target planes in the reference volume in accordance with a pre-determined scanning protocol. For example, a set of standard cardiac views may be targeted.

At step 704 of the method, an ultrasound imaging procedure is performed, at a later time from the time at which step 702 was performed. The ultrasound imaging procedure guides an operator to perform scans of the N desired target planes that have been selected.

At step 706, a registration between the reference volume and the live ultrasound is performed. This registration can be performed manually or automatically. In further detail, a manual registration may be performed by browsing/rotating the volume to show a specific anatomic plane (analogous to the easy landmark plane), then finding the same anatomic plane in the patient on the live ultrasound, and then use some user interaction (e.g. via the user interface) to say the volume is now registered, The registration may be automatic, for example, using scanning a new 3D ultrasound volume and using an algorithm to register the new volume with the previously acquired 3D volume.

In further embodiments, a record of the scanning process is stored (for example, the position/orientation data and the images scanned). This record allows the scanning method to be reproduced or studied at a later date.

Certain embodiments provide a method of guiding performance of an ultrasound imaging procedure, comprising: detecting a position of an ultrasound probe; determining a position of an imaging region of the ultrasound probe relative to a target based on the position of the ultrasound probe; displaying a figure onto an image, wherein the figure has information corresponding to a distance between the position of the imaging region and a position of an imaging plane corresponding to the image; and updating the appearance of the figure as the ultrasound probe moves relative to the target.

Displaying at least one indicator may show the position of the ultrasound probe corresponding to the target position. The figure may comprise a representation (optionally a fan representation) of a boundary of the imaging region. The representation may comprise a fan representation that represents an outline of a projection of a 2D or 3D scan plane scanned by the probe. The method may comprise varying appearance (optionally, colour, size and/or texture) of the figure in dependence on the position and/or orientation of the probe or the imaging region relative to the target.

The figure may comprise a representation (optionally a fan representation) of a boundary of the imaging region, and the varying of the appearance comprises varying at least one of: a) colour or line appearance (for example, solid, dashed or dotted) of at least part of the representation, optionally colour of a boundary line; b) colour and/or position and/or shape and/or size of at least one marker positioned on or relative to the boundary.

The colour of at least part of the representation, and/or said colour and/or position and/or shape and/or size of at least one marker, may vary at different positions on or relative to the boundary. The at least one marker or each marker may comprise at least one of a rectangular marker, a square marker, a round marker, a circular marker or other regular-shaped marker, and/or an error bar. The method may comprise displaying the figure on a display screen together (for example, overlaid) with an image of the subject, optionally a current or previously-generated image such as an ultrasound image.

The method may comprise displaying at least two windows, wherein a first one of the windows displays the indicator, and a second one of the windows displays the figure. The indicator may be updated in real time as the ultrasound probe moves relative to the target. The target may comprise a target position of the probe and/or a target plane. The method may comprise displaying an indication of similarity of an image or part of an image produced by the probe (optionally an image plane) to a target image (optionally a target plane). The target may comprise a target identified using a previous imaging procedure and/or other imaging modality. The image may be a tomographic image obtained by medical imaging apparatus. The imaging region may have a plurality of corners and the figure shows each information corresponding to a distance between the position of the imaging region and a position of an imaging plane corresponding to the image at least two corners of the imaging region.

Certain embodiments provide an apparatus to guide a user where on the body to place an ultrasound probe to scan a specific anatomical plane or structure, comprising a display showing: representations of the live and target probes in different colours; the representations showing the position and orientation offset of live from target probe; and the probe representations updated in real-time as the user moves the probe. A first representation may be realistic 3D models of the probes, rendered in their correct relative position and orientation. A second representation may be a more symbolic and allow for separate visualisation of the angular and translational offset of live and target probes to facilitate final fine-tuning of the offset. The display may switch dynamically between the first representation and the second representation depending on whether the translation offset between live and target probes is above or below a threshold.

The target probe may be shown fixed, whilst the position and orientation of the live probe is updated in real time. Additional guidance may be provided by colouring the ultrasound fan, where: the fan has one colour when the live and target planes are aligned and/or the fan has different colour(s) when the live plane is infinitely far from the target plane. The fan may represent the outline of the 2D scan plane scanned by a 2D probe. The fan may represent the outline of the 3D scan volume scanned by a 3D probe. The fan may be green when the live and target planes are aligned. The fan may be a second colour (e.g. red) when the live plane is infinitely far behind the target plane. The fan may be a third colour (e.g. blue) when the live plane is infinitely far in front of the target plane. The fan colour may vary continuously around its perimeter between these three colours based on the signed distance, perpendicular to the display, between the live scan plane and the target scan plane. The fan may be dashed when the live plane is flipped compared to the target plane, such that their normals are at a mutually obtuse angle.

Additional guidance may be provided by adding markers spaced around the border of the ultrasound fan, where: the size of the markers is based on the magnitude of the distance, perpendicular to the display, between the live scan plane and the target scan plane; the size of the markers may increases as this distance increases; the size of the markers falls to zero when this distance is zero. The markers may be rectangular. The markers may be circles. The markers may take the form of an error-bar. The marker colour is different depending on whether the current scan plane is behind or in front of the target plane.

Additional guidance is provided by displaying a similarity between the actual and target Ultrasound images. The similarity can be based on a simple image metric or, where the target plane is a ‘standard’ e.g. cardiac plane, on an algorithm that has been trained on prior images of the target and surrounding planes. The plane to be scanned is that scanned in a prior exam of the same patient.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms and modifications as would fall within the scope of the invention.

1. A method of guiding performance of an ultrasound imaging procedure, comprising:
detecting at least a position of an ultrasound probe;
determining a position of an imaging region of the ultrasound probe relative to a target based on the at least detected position of the ultrasound probe;
displaying a figure representative of an imaging region together with a reference image associated with the target wherein the appearance of at least part of the figure is dependent on at least the determined position of the imaging region relative to the target; and
updating the appearance of at least part of the figure as the ultrasound probe moves relative to the target.

2. The method according to claim 1, wherein displaying the figure comprise positioning or orienting the figure relative to the reference image such that the position and/or orientation of the figure relative to the reference image is representative of a position and/or orientation of the imaging region relative to the target.

3. The method according to claim 1, wherein displaying the figure comprises overlaying the figure onto the reference image.

4. The method according to claim 1, wherein the figure comprises a representation of a boundary of the imaging region.

5. The method according to claim 1, wherein the figure comprises a representation of an outline of a projection of a 2D scan plane or 3D scan region scanned by the probe.

6. The method according to claim 1, wherein the figure comprises at least one marker positioned on or relative to a boundary of the imaging region.

7. The method according to claim 6, wherein the at least one marker or each marker comprises at least one of a rectangular marker, a square marker, a round marker, a circular marker or other regular-shaped marker, and/or an error bar.

8. The method according to claim 1, wherein updating the appearance of at least part of the figure comprises varying the appearance of at least part of the figure in response to a change in one or more of: the position and/or orientation of the probe and/or the position and/or orientation of the imaging region relative to the target.

9. The method according to claim 1, wherein varying the appearance of at least part of the figure comprises varying at least one visual aspect of at least part of the figure.

10. The method according to claim 9, wherein the at least one visual aspect comprises at least one of: colour, size and/or texture.

11. The method according to claim 4, wherein varying the appearance of at least part of the figure comprises varying at least one visual aspect of at least part of the figure, and wherein the at least one visual aspect comprises a line appearance of the boundary of the imaging region, optionally wherein varying the line appearance comprises changing the line appearance to be one of solid, dashed or dotted and/or changing the colour of the line.

12. The method according to claim 6, wherein varying the appearance of at least part of the figure comprises varying at least one visual aspect of at least part of the figure, and wherein the at least one visual aspect comprises the colour and/or position and/or shape and/or size of at least one marker and/or each of the at least one marker.

13. The method according to claim 9, wherein varying the at least one visual aspect comprises a continuous variation of the at least one visual aspect dependent on the position of the imaging region relative to the target.

14. The method according to claim 9, wherein varying the at least one visual aspect comprises varying at least one visual aspect of a first part of a figure to a first degree and varying the at least one visual aspect of a second part of the figure to a second, different degree.

15. The method according to claim 1, wherein at least one of: the reference image comprises an image of a subject, wherein the image of the subject comprises a previously-generated image.

16. The method according to claim 1, wherein the figure is displayed together with at least one further indicator comprising a representation of the probe and/or a representation of the target.

17. The method according to claim 1, wherein the at least one further indicator comprises a first and second indicator, wherein the first indicator is representative of an orientation of the imaging region relative to the target and the second indicator is representative of the position of the imaging region relative to the target.

18. The method according to claim 16, wherein the method further comprises displaying the at least one further indicator in response to a measure of distance between the imaging region and the target being below a pre-determined threshold.

19. The method according to claim 1, wherein at least one of a) and b):
a) the display is updated in real time as the ultrasound probe moves relative to the target;
b) the appearance of the figure is updated while the reference image is fixed in response to movement of the ultrasound probe.

20. The method according to claim 1, wherein the target comprises a target position of the probe and/or a target plane.

21. The method according to claim 1, wherein the figure adopts a first appearance when the imaging region is in front of the target and a second appearance when the imaging region is behind the target.

22. The method according to claim 1, further comprising determining an indication of similarity between an image or part of an image produced by the probe to the reference image and displaying the indication of similarity.

23. The method according to claim 1, wherein the target comprises a target identified using a previous imaging procedure and/or other imaging modality and/or wherein the reference image comprises a tomographic image obtained by a medical imaging apparatus.

24. An apparatus comprising processing circuitry configured to:
receive position data representative of at least a detected position of an ultrasound probe;
determine a position of an imaging region of the ultrasound probe relative to a target based on detected position of the ultrasound probe;
display a figure representative of the imaging region together with a reference image associated with said target, wherein the appearance of at least part of the figure is dependent on at least the determined position of the imaging region relative to the target; and
update the appearance of at least part of the figure as the ultrasound probe moves relative to the target.

25. A computer program product comprising computer-readable instructions that are executable to:
receive position data representation of at least a detected position of an ultrasound probe;
determine a position of an imaging region of the ultrasound probe relative to a target based on the detected position of the ultrasound probe;
display a figure representative of an imaging region together with a reference image associated with the target wherein the appearance of at least part of the figure is dependent on at least the determined position of the imaging region relative to the target; and
update the appearance of at least part of the figure as the ultrasound probe moves relative to the target.
